(19) United States
(12) Patent Application Publication
(10) Pub. No.: US 2004/0106964 A1
(43) Pub. Date: Jun. 3, 2004

(54) Implantable Medical Device with Multiple Electrode Lead and Connector with Central Fastener

(76) Inventors: Elmar R. Fischer SR., Centerville, MN (US); John A. Schmidt, Leesburg, VA (US)

Correspondence Address: JOHN RICHARD MERKLING, 11171 WEST EXPOSITION DIRVE, LAKEWOOD, CO, 80226, US

(21) Appl. No.: 10/605,529

(22) Filed: Oct. 6, 2003

Related U.S. Application Data
(60) Provisional application No. 60/319,606, filed on Oct. 10, 2002.

(51) Int. Cl. A61N 1/375

(52) U.S. Cl. 607/36

(57) ABSTRACT

An implantable medical device such as a cardiac stimulator, a multi-electrode lead attached to the device, and a connector coupling the device to the lead. The lead has multiple electrodes, each electrode connected to a wire extending though the lead. The electrodes may be circumferential coils or rings, for example. The lead has a connector that fits into a recess on a surface of the device or apparatus. A bottom wall of the recess has an array of apparatus connections deployed around a threaded bore. The connector is attached to the apparatus by a screw with a threaded shaft and an enlarged head. The screw passes through a central bore in the connector. Electrical connections form a regular pattern, such as a rectangular or square grid, or a radial pattern, around the central bore. A pair of O-rings or seals surround the connections. A gasket, mounted on male connections or contacts, fits around female connections that may be on either the apparatus or the connector.

### BACKGROUND OF INVENTION

This invention pertains to cardiac stimulation devices such as pacemakers, cardiovertors and defibrillators and particularly to devices using multiple electrodes, and more particularly, to connectors for coupling multiple electrode leads to implantable devices.

The heart is a mechanical pump that is stimulated by electrical impulses. The mechanical action of the heart results in the flow of blood. During a normal heartbeat, the right atrium (RA) fills with blood from the returning veins. The RA then contracts and this blood is moved into the right ventricle (RV). When the RV contracts it pumps that blood to the lungs. Blood returning from the lungs moves into the left atrium (LA), and after LA contraction, is pumped into the left ventricle (LV), which then pumps it throughout the body. Four heart valves keep the blood flowing in the proper directions.

The electrical signal that drives this mechanical contraction starts in the sino-node, a collection of specialized heart cells in the right atrium that automatically depolarize (change their voltage potential). This depolarization wave front passes across all the cells of both atria and results in atrial contraction. When the advancing wave front reaches the A-V node it is delayed so that the contracting atria have time to fill the ventricles. The depolarizing wave front then passes over the ventricles, causing them to contract and pump blood to the lungs and body. This electrical activity occurs approximately 72 times a minute in a normal individual and is called normal sinus rhythm.

The corresponding electrical signals identifying these events are usually referred to as the P, QRS (or R) and T waves or beats. More particularly, an atrial contraction is represented on an ECG by a P wave, a ventricular contraction is represented by an R wave and a ventricular repolarization is represented by a T wave. The atrium also repolarizes but this event (the U wave) is masked by activity in the ventricle and consequently it is not observable on an ECG.

Conventional pacemakers utilize a single or dual leads to apply pacing pulses. The dual (bipolar) lead typically includes a tip and a ring electrode. The lead is inserted in such a manner that the tip is imbedded into the cardiac muscle. A pacing pulse is then applied between the tip and the ring electrodes, thereby causing the cardiac muscle to contract. If a single unipolar electrode lead is used, the electric pulse is applied between the tip electrode and another electrode outside the heart, for example, the housing of the pacemaker. Bradycardia pacing therapy has usually been delivered through a pacing electrode implanted near the ventricular apex, that is, near the bottom of the heart. This location has been preferred not for physiologic reasons, but because most lead designs favor implantation at this site. A lead entering the right ventricle from the right atrium tends to extend into the lower apex of the ventricle where an active fixation apparatus, such as a helical corkscrew, may be used to secure the lead to the heart wall. Even if the distal tip of the lead is implanted at another location, it may be difficult or impossible to move the electrode to another location within the heart after initial implantation. The physician is thus limited to a single site for applying treatment. Bradycardia pacing therapy can be improved by delivering the stimulating pulse to a more efficient location than the ventricular apex. Studies have indicated that the abnormal contraction that results from apical pacing has long-term deleterious effects. Short-term studies using conventional pacing leads implanted in alternative locations have shown clinical improvements, but the long-term reliability of conventional pacing leads in these alternative locations is questionable and lead placement is difficult.

A single stimulating electrode, such as one available on a conventional lead, may not be implanted close enough to a physiologically preferred location in the patient's heart to cause improved cardiac efficiency when the pacemaker stimulates the heart. In fact, stimulating at the bottom end of the ventricle may diminish cardiac efficiency as compared to a wave propagated from the top of the ventricle. Moreover, an apparatus with a single electrode cannot control cardiac contraction, guide the propagation of a wave front, force a selected path for a stimulating wave front, or create a coordinated simultaneous or near simultaneous cardiac contraction of large sections of the myocardium. Such controlled contractions may result in more efficient cardiac contraction, thereby reducing the overall demand on the heart, allowing the body to alleviate the symptoms associated with inefficient blood flow. There is a need, therefore, for implantable devices, specifically cardiac stimulators, which can utilize a relatively large number of electrodes to stimulate and sense the heart in multiple locations.

Multiple electrode leads, however, are difficult to connect to implantable devices. The standard connector technology used in implantable medical devices stacks electrical contacts and insulators one after the other in a linear manner. Consequently, the manufacturing tolerances, and errors, accumulate. After a few electrical contacts, the position of electrical contacts cannot be assured to within acceptable manufacturing limits. Current technology for pacemaker lead connectors was developed about forty years ago and has become embodied in certain standard connectors. In general, however, such standard connectors are limited to one (“unipolar”) or two (“bipolar”) electrodes.

It has been suggested that similar linear connectors may be used in multiple electrode leads. Harris, for example, has suggested such configurations in several patents, such as U.S. Pat. No. 4,715,380 and U.S. Pat. No. 4,995,389. Such connectors rapidly reach practical limits to the number of electrodes and connections that can be accommodated.

### SUMMARY OF INVENTION

In view of the above disadvantages of the prior art, it is an objective of the present invention to provide an implantable cardiac stimulation system, such as a pacemaker, in which three or more electrodes are positioned in a chamber of the heart and a connector that can couple an implantable medical device and a multi-electrode lead. The connector can accommodate a large number of separate connections, for example, thirty-two, sixty-four or one hundred and twenty-eight connections.

It is an object of the present invention to provide an implantable cardiac stimulator system or medical apparatus comprising a cardiac stimulator having means for stimulating the heart of a patient, a hermetically sealed case, and a multiple electrode lead, the lead being coupled to the medical apparatus by a multiple conductor connector.

A further object of the invention is to provide a multiple conductor connector for a multiple electrode medical device wherein the connector is secured by a single screw.

Another object of the invention is to provide a multiple conductor connector with multiple redundant seals.

A further object of the invention is to provide a multiple conductor connector with independent connections such that manufacturing tolerances may be maintained.

It is also an object of the invention to provide a connector for a multi-electrode lead having a lumen opening into the lead such that a stylet can be inserted through the lumen into the lead.

Other objectives and advantages of the invention will become apparent from the following description.

Briefly, the subject invention pertains to an implantable medical device such as a cardiac stimulator, a multi-electrode lead attached to the device, and a connector coupling the device to the lead. The term cardiac stimulator will be used herein to cover pacemakers as well as other cardiac devices such as cardioversion devices and defibrillators. The lead is inserted in the body into an organ to be sensed or stimulated, for example, into a cardiac chamber. Alternatively, the lead may be positioned in the veins, or it may be positioned externally of the heart or other organ to be stimulated or sensed. Since the lead has many electrodes, a multiple connector must be provided to couple the device to electrodes or sensors in the lead.

In a preferred embodiment, a lead having an elongated member is provided with the electrodes being formed on the elongated member. The electrodes comprise axially spaced electrodes disposed on the elongated member, each electrode being connected by a wire extending though said elongated member. The electrodes may be circumferential coils integral or continuous with the wires or may be rings connected to the wires by crimping or laser welding, for example. An electrode may also be provided at the distal end of the lead. The elongated member may be a tube housing the wires. The electrodes can be angularly spaced with respect to each about the elongated member. The tube may include an elongated cavity adapted to receive a removable stylet. The stylet may be more rigid then the lead and may be used for the implantation of the lead. After the lead is implanted, the stylet is removed.

The lead has a connector that fits into a recess on a surface of the device or apparatus. A bottom wall of the recess has an array of apparatus connections deployed around a threaded bore. The connector is attached to the apparatus by a screw with a threaded shaft and an enlarged head. The screw passes through a central bore in the connector. Electrical connections form a regular pattern, such as a rectangular or square grid, or a radial pattern, around the central bore. A pair of O-rings or seals surround the connections. A gasket fits around female connections that may be on either the apparatus or the connector

### BRIEF DESCRIPTION OF DRAWINGS

FIG. 1 shows a diagrammatic front view of a patient with an implantable device, specifically, a cardiac stimulation system.

FIG. 2 is a cross-sectional view of a heart with an implanted lead with a multiple conductor connector.

FIG. 3 is a plan view of a coil electrode.

FIG. 4 is a cross sectional view of a ring electrode.

FIG. 5 is a cross section of the multi-electrode lead of FIG. 3, taken along line 5-5.

FIG. 6 is a perspective view of an implantable medical apparatus, specifically, a cardiac stimulation system, with a lead connector.

FIG. 7 is an exploded perspective view of the apparatus and connector of FIG. 6.

FIG. 8 is an exploded perspective view of the connector of FIG. 7.

FIG. 9 is an exploded cross sectional view of the connector of FIG. 8, taken along line 9-9.

FIG. 10 is a perspective view of a first embodiment of a female connection, used in the connector.

FIG. 11 is a cross sectional view of the female connection of FIG. 10, taken along the line 11-11.

FIG. 12 is a perspective view of a second embodiment of a female connection, used in the connector.

FIG. 13 is a cross sectional view of the female connection of FIG. 12, taken along the line 13-13.

FIG. 14 is a partial cross sectional view of a header area of the implantable medical apparatus of FIG. 7, taken along line 14-14.

FIG. 15 is a partial cross section of a back portion of the header area of FIG. 14.

FIG. 16 is a partial cross section of male connections on a front portion of the header area of FIG. 14.

FIG. 17 is a plan view of the male connections, illustrating a configuration of thirty-two connections.

FIG. 18 is a plan view of the male connections, illustrating a configuration of sixty connections.

FIG. 19 is a plan view of a pin for a male connection.

FIG. 20 is a plan view of a second embodiment of a pin for a male connection.

FIG. 21 is a perspective view of a gasket used in the header area of FIG. 14.

FIG. 22 is a is a partial cross sectional view of the assembled connector and header area of the implantable medical apparatus of FIG. 6, taken along line 22-22

### DETAILED DESCRIPTION

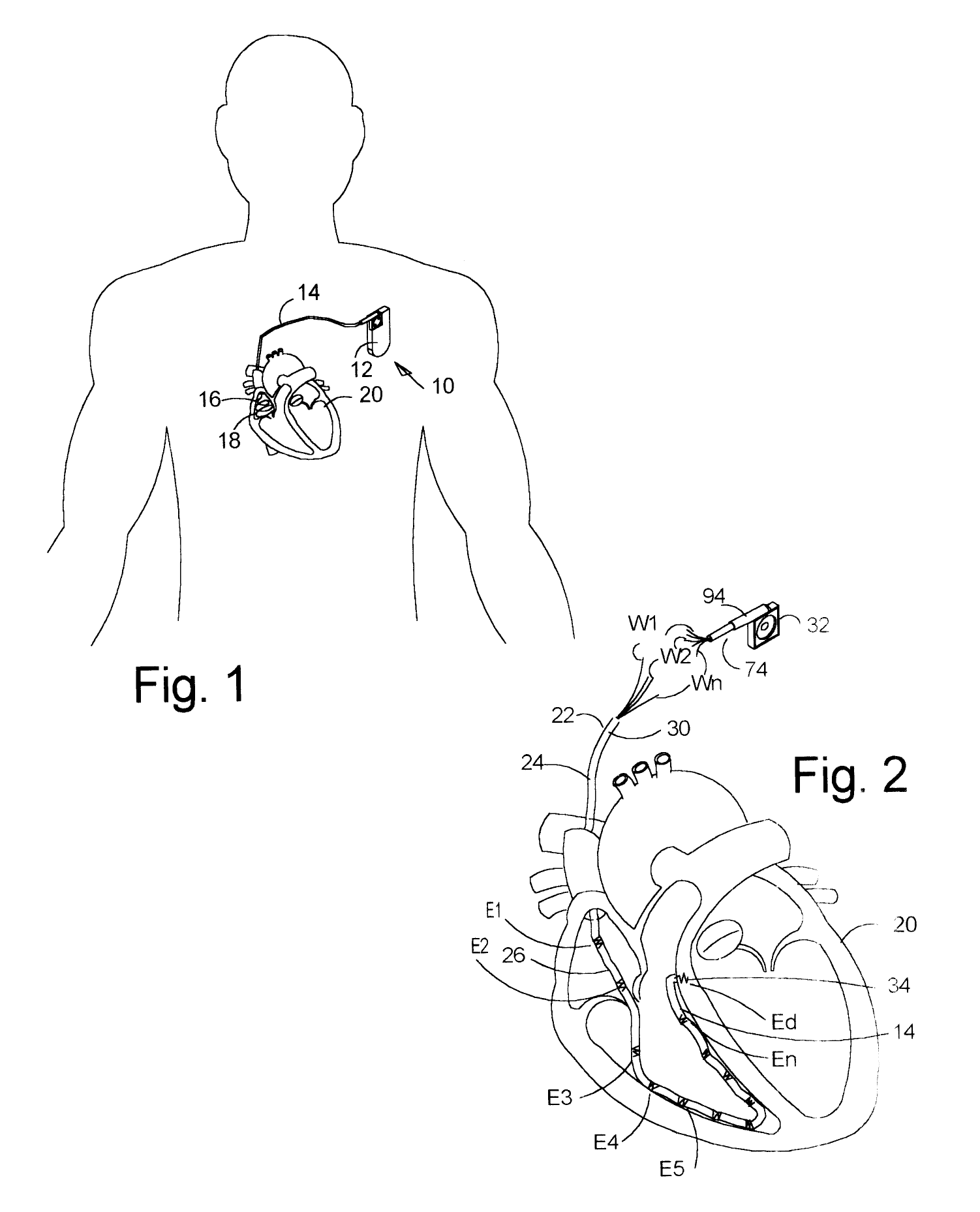

The subject invention pertains to an implantable medical device, such as a cardiac stimulation system 10 including a cardiac stimulator 12 with various electronic circuits, and a multi-electrode lead 14 attached to the stimulator 12, as shown in FIG. 1. The lead 14 has a distal end 16 disposed, for example, in one of the cardiac chambers, such as the right atrium 18 of heart 20. In FIG. 1, end 16 is shown having a general spiral shape. An alternative configuration is shown in FIG. 2. Numerous configurations may be used without departing from the teachings of the invention. The system 10 is adapted to deliver therapy in the form of electrical pulses. The therapy may include GCV (greater cardiac vein) resynchronization therapy, treatment of conduction pathway abnormalities, bardycardia pacing, and other therapies. The cardiac stimulator 12 contains electronic components common to current cardiac stimulators such as a battery, microprocessor control circuit, ROM, RAM, an oscillator, reed switch and antenna for communication, output circuits, and sense circuits. These components are well known to those of skill in the art. In addition, the cardiac stimulator 12 has a plurality of independent sensing and stimulating circuits for each heart chamber. Further details related to appropriate multi-channel sensing and stimulating circuits are found in commonly assigned U.S. patent application Ser. No. 10/134,197, filed Apr. 26, 2002, the disclosure of which is incorporated herein by reference.

Multi-Electrode Lead

Details of the multi-electrode lead 14 are shown in FIGS. 2 through 5. The lead 14 includes an external biocompatible polymer tube 22 having a straight portion 24 and a shaped portion 26. The tube may be made of polyurethane or other similar materials that may be thermally shaped so that the shaped portion 26 retains any desired configuration. In FIG. 1, the shaped portion 26 is shown as having a spiral shape, but many other shapes, such as the curved shape shown in FIG. 2, may be selected as well. The spiral or coil-shaped lead of FIG. 1 and curved shape of FIG. 2 places electrodes around an entire chamber of the heart. Such embodiments allow complete sensing and stimulating control around the entire chamber. Nevertheless, it will be apparent that numerous shapes could be selected to address the clinical needs of a particular patient.

A plurality of electrodes E1, E2, E3, E4, E5, . . . En are attached to tube 22 of the lead 14. Preferably electrodes E1 . . . En are formed of coils of bare wire or cable wound about the tube 22. Each electrode is connected to corresponding wires W1, W2, W3 . . . Wn which extend through the length of tube 22 and which are shown exiting through end 30 for the sake of clarity. Wires W1, W2, W3 . . . Wn are insulated, so that they are not shorted to each other within the tube 22. The electrode 14 and its method of manufacture are disclosed in co-pending commonly assigned application Ser. No. 09/245,246, incorporated herein by reference. Preferably the end 30 of tube 22 and the ends of wires W1, W2, W3, . . . Wn are coupled to a connector 32 for attaching the lead 14 to the cardiac stimulator 12. The connector 32 may have a plurality of connections, as explained hereafter. Male or female connections may be placed on the connector 32 and corresponding female or male connections may be placed on the medical apparatus. In the preferred embodiment, male connections are mounted on the medical apparatus, as this configuration is conducive of forming an hermetic seal for the medical apparatus, as explained below. Each wire W1 . . . . Wn is associated with a connection.

In addition to spiral coil or ring electrodes E1 . . . En, a distal tip electrode Ed may also be provided. The distal tip electrode Ed may also have an active fixation mechanism, for example a helical screw 34 or tines, to secure the lead to the interior wall of the heart. The lead 14 can be constructed with the tube 32 extending relatively straight or can be customized to any shape to fit any pre-selected location within the heart 20, dependent on each particular patient's pathology. For example, if the lead 14 is to be placed in the greater cardiac vein, then its end 16 (consisting of tube portion 26 and electrodes E1, E2, E3 . . . En.) is shaped to form a small helix, so that it will fit into the great cardiac vein.

The tube 22 can be formed with a longitudinal cavity 36, as shown in the cross sectional view of FIG. 5. Cavity 36 holds the wires W1, W2, W3 . . . . Wn. The lead 14 could be straightened by inserting a substantially straight stylet 40 into an interior tube or lumen 42. The stylet 40 is also flexible but is less flexible than the lead 14, so that as the stylet is inserted into the lumen 42, it forces the tube 22 to straighten. The lead 14 is then inserted into the heart or into a vein near the heart. After implantation of the lead 14, the stylet 40 is withdrawn and the lead 14 flexes back towards the original configuration of the lead.

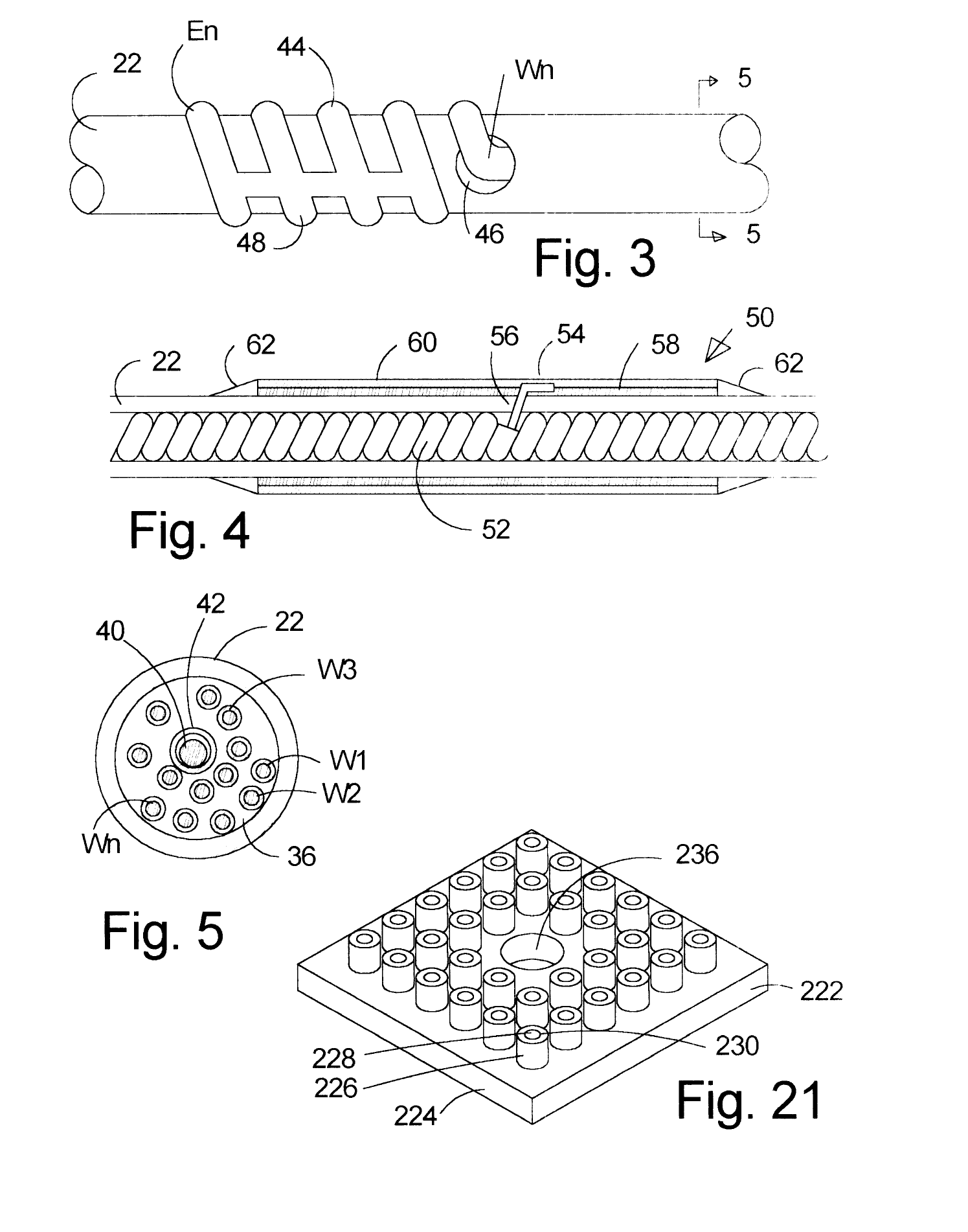

A plurality of electrodes E1, E2, E3, E4, E5, . . . En are attached to tube 22 of the lead 14. Preferably electrodes E1 . . . En are formed of coils 44 of exposed wire or cable wound about the tube 22, as shown in FIG. 3. The wire Wn passes through a predrilled hole 46 in the tube 22. The predrilled hole 46 determines the exact location of the electrode. By changing the position and spacing of the holes, leads may be designed to cluster more electrodes along a selected segment of the lead. Since the electrodes fully circumvent the tube 22, it is likely that at least some part of the electrode will be adjacent the cardiac wall. Moreover, circumferential electrodes are unlikely to perforate the heart. Preferably the coil 44 and wire Wn are formed of one continuous wire. The loops of the coil 44 are welded 48 or otherwise connected together to provide additional structural stability. Each electrode is connected to corresponding wires W1, W2, W3 . . . Wn which extend through the length of tube 22 and which are shown exiting through end 30 for the sake of clarity. Wires W1, W2, W3 . . . Wn are insulated, so that they are not shorted to each other within the tube 22. The lead 14 is more particularly disclosed in co-pending commonly assigned application Ser. No. 09/761,333, incorporated herein by reference. Preferably the end 80 of tube 72 and the ends of wires W1, W2, W3 . . . . Wn are coupled to a connector 32 for attaching the lead 14 to the cardiac stimulator 12. The connector 32 may have a plurality of connections, as more fully described below. Each one of the wires W1 . . . Wn is associated with a connection.

An alternative configuration for an electrode 50 is illustrated in FIG. 4. In this configuration, a multi-filar coil 52 comprises as many insulated-wires as there are electrodes on the lead. The multi-filar coil 52 lies within the tube 22. At a location of an electrode 50, an end 54 of one of the wires passes through a hole 56 in the tube 22 and contacts an inner ring 58. A hole may also be provided in the inner ring for the wire or two inner rings may be used, one ring on either side of the wire. An outer ring 60 is placed over the inner ring or rings and crimped, capturing the end 54 of the wire between the inner and outer rings. The electrical and mechanical connection between the rings and the wire may also be improved by welding or other methods. A circumferential bead 62 of glue may seal the ends of the rings and reduce sharp edges.

An alternative configuration for an electrode 50 is illustrated in FIG. 4. In this configuration, a multi-filar coil 52 comprises as many insulated-wires as there are electrodes on the lead. The multi-filar coil 52 lies within the tube 22. At a location of an electrode 50, an end 54 of one of the wires passes through a hole 56 in the tube 22 and contacts an inner ring 58. A hole may also be provided in the inner ring for the wire or two inner rings may be used, one ring on either side of the wire. An outer ring 60 is placed over the inner ring or rings and crimped, capturing the end 54 of the wire between the inner and outer rings. The electrical and mechanical connection between the rings and the wire may also be improved by welding or other methods. A circumferential bead 62 of glue may seal the ends of the rings and reduce sharp edges.

Multi-Conductor Connector

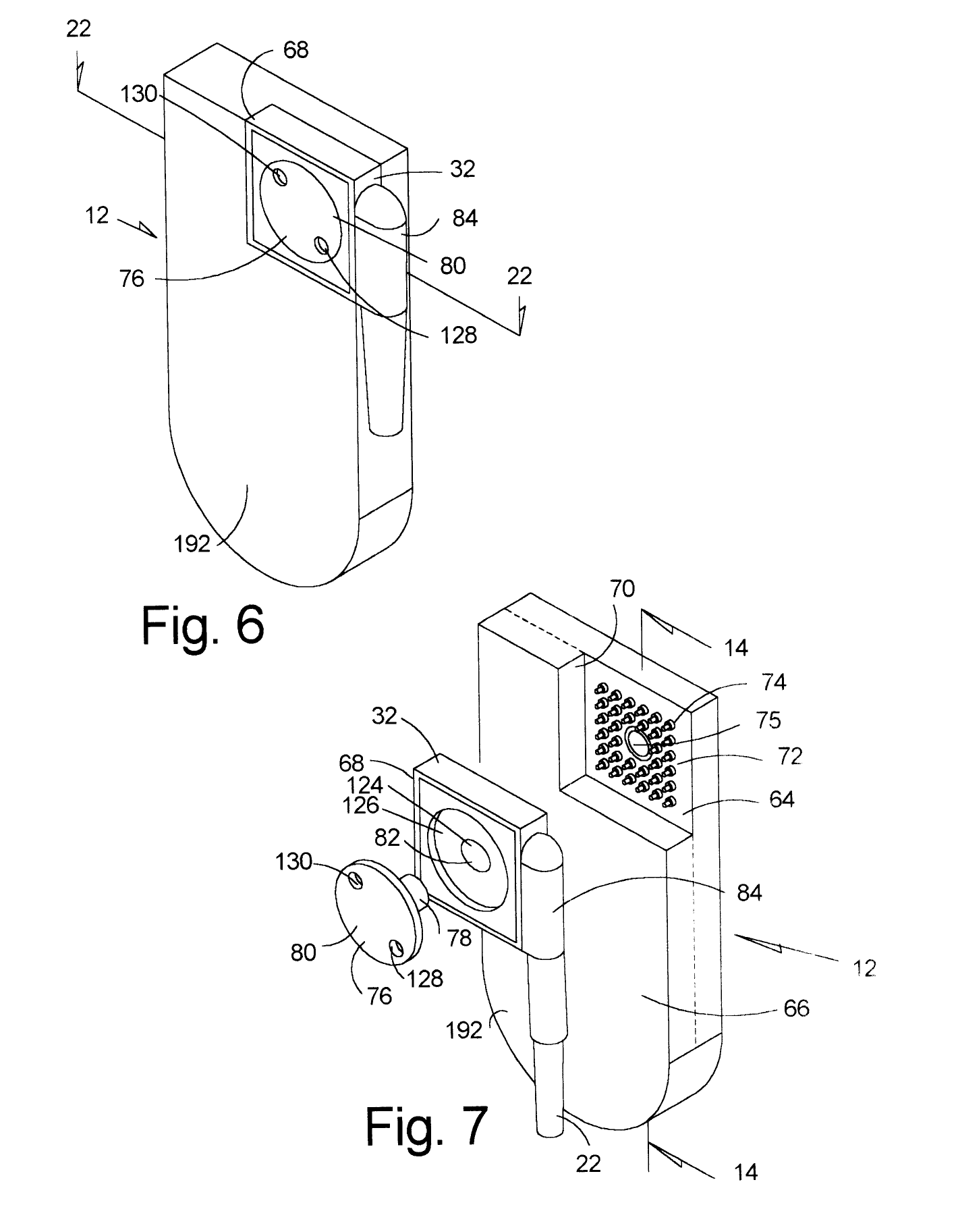

FIG. 6 shows the multi-conductor connector 32 mounted on an implantable device 12, such as a cardiac stimulator. An exploded view of the device 12 and the connector 32 is shown in FIG. 7. The connector 32 preferably fits into a recess 64 on a surface 66 of the device or apparatus 12. A peripheral wall 68 of the connector 32 conforms to a side wall 70 of the recess 64. These walls may have any suitable configuration, such as polygonal, rectangular, or curved. The connector may also be attached directly to the surface 66 without a recess 64. A recess is preferred, however, because it provides a lower profile for the apparatus 12 and connector and because the contact of the peripheral wall 68 and the side wall 70 resists rotation.

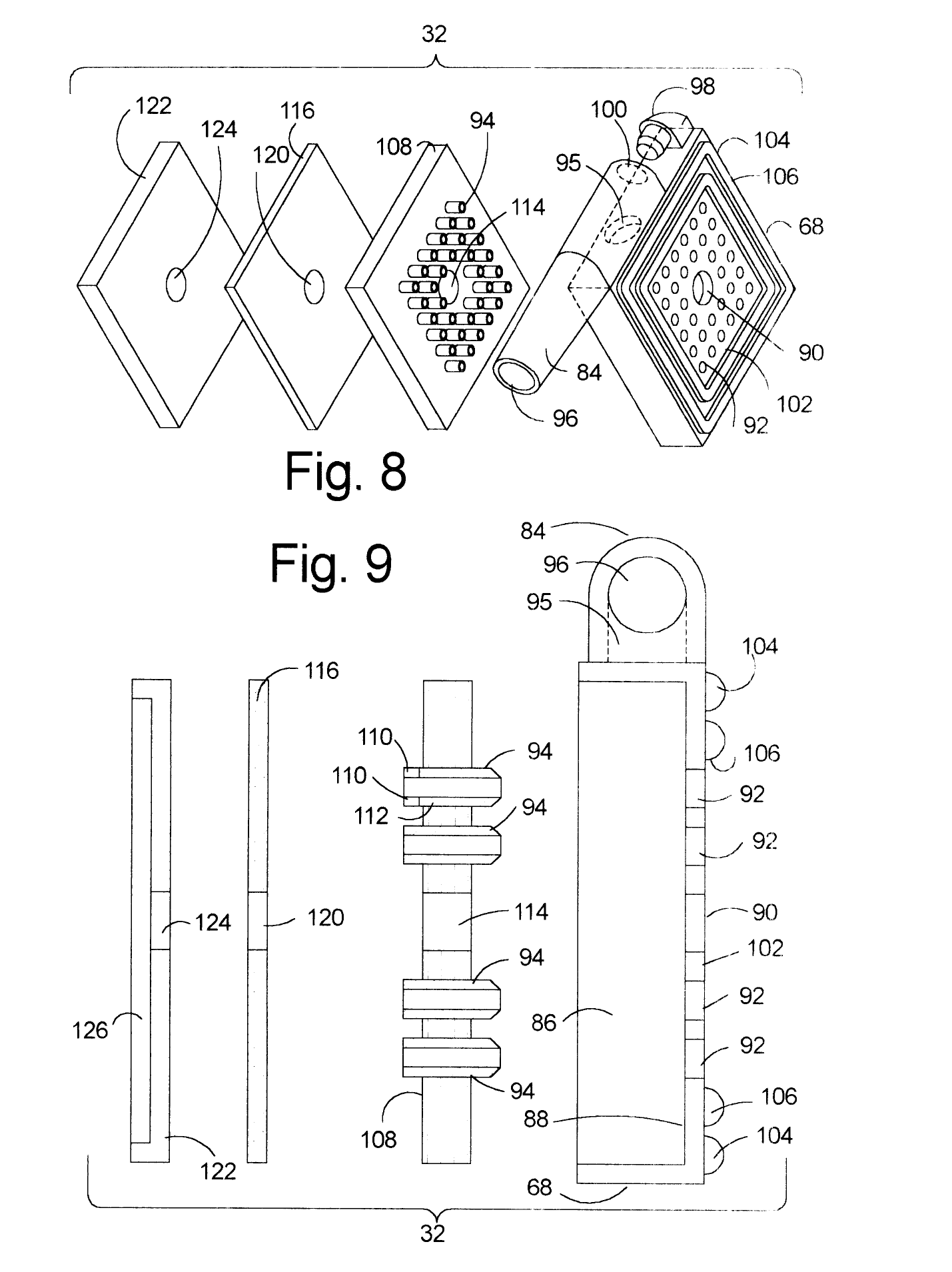

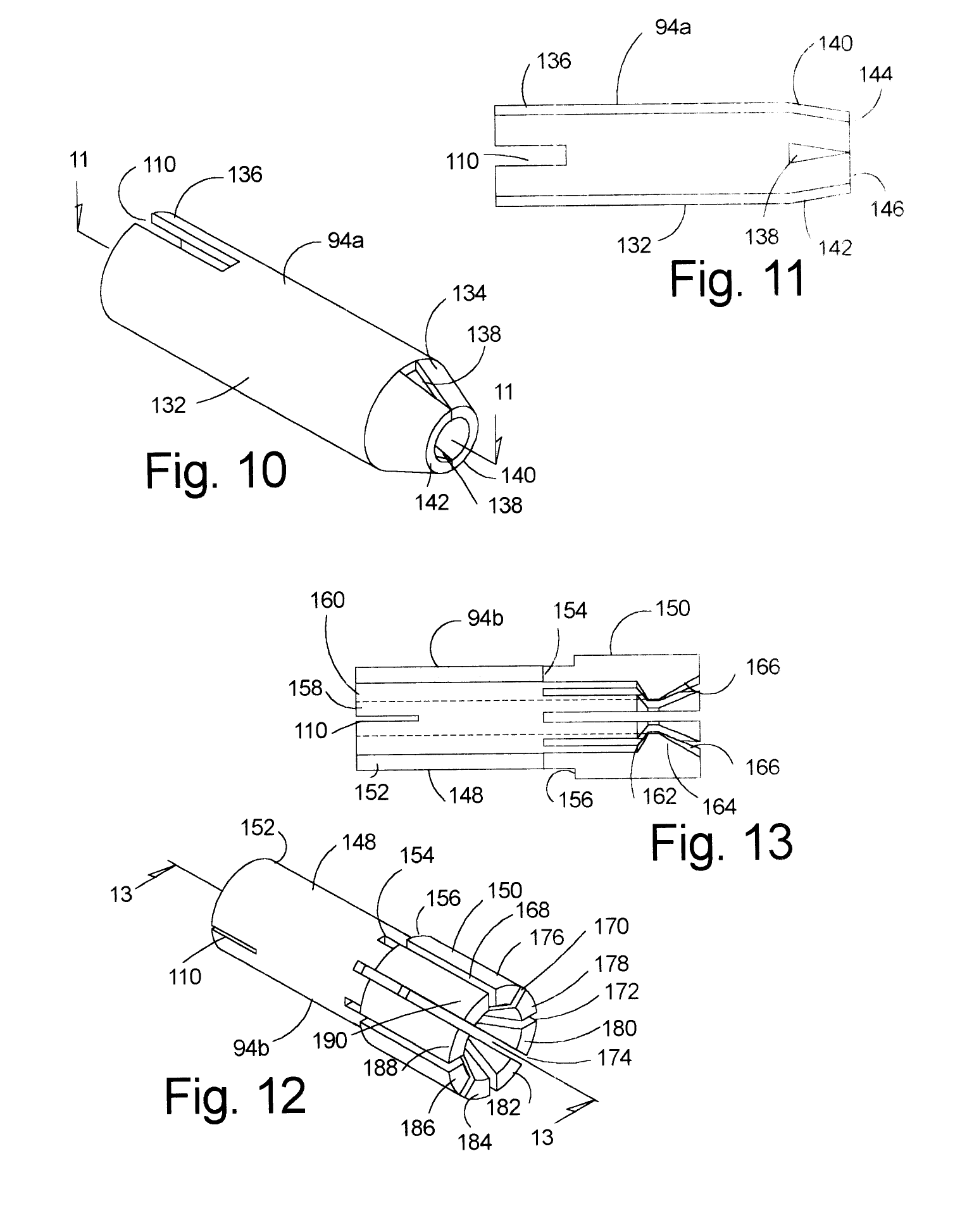

A bottom wall 72 of the recess 64 has an array of apparatus connections 74 deployed around a threaded bore 75. In the preferred embodiment, the apparatus connections 74 are pins, as more fully explained below. Corresponding connections are found on the connector 32. The connector 32 is attached to the apparatus 12 by a screw 76 with a threaded shaft 78 and an enlarged head 80. The threaded shaft 78 passes through a central bore 82 in the connector 32 and engages the threaded bore 75 in the apparatus. A strain relief tube 84 mounted on the connector 32 couples the lead body 22 and the connector 32. Further details of the connector 32 can be seen in FIGS. 8 and 9.

The connector 32 comprises a box 86 having a floor 88 and peripheral wall 68. In the preferred embodiment, the floor 88 is generally square, but other configurations or shapes may be selected. The floor 88 has a central bore 90 for receiving the screw 76. A plurality of holes 92 are distributed around the central bore 90. Connections 94, described in greater detail hereafter, pass through the holes 92. The configuration of the holes 92 and connections 94 is preferably a regular pattern, such as a rectangular or square grid, or a radial pattern. The strain relief tube 84 is mounted on the peripheral wall 68. An opening 95 through the wall 68 provides a path for the wires W1 . . . Wn through a central lumen 96 in the strain relief tube 84 into the lead 22. A removable plug 98 seals a proximal end 100 of the lumen. When the plug is temporarily removed, the sylet 40 can be inserted into the lead, as described above. An outer side 102 of the floor 88 conforms to the bottom wall 72 of the recess. On the outer side 102 of the floor, a pair of O-rings or seals 104, 106 surround the holes 92 and connections 94. The seals 104, 106 preferably follow the peripheral wall 68.

A non-conductive plate 108 fits within the box 86 and supports the connections 94. The plate 108 may be a material such as Mylar. In the preferred embodiment, the connections 94 are female connections, but male pins could also be used in the connector 32, while female connections could be used on the apparatus 12. The wires W1 . . . Wn are connected to the connections by mechanically inserting ends of the wires in slots 110 in sides 112 of the connections 94, by welding, or by other suitable means. A central bore 114 in the plate 108 corresponds to the central bore 90 in the box and provides a passageway for the screw 76. The wires W1 . . . Wn extend through the opening 95 and into the lead 22. With the plate 108 positioned in the box 68, a gasket 116 is placed over the plate 108. The gasket 116 fits over the female connections 94 and the compressible gasket allows the wires W1 . . . Wn to extend from the connections to the lead. A third central bore 120 corresponds to the central bore 114 in the plate 108 and provides a passageway for the screw 76. Behind the gasket 116, a pressure plate 122 fits into the box 86 over the gasket 116. A fourth central bore 124 provides a passageway for the screw 76. A recess 126 surrounds the central bore 124 and allows the head 80 of the screw 76 to be tightened essentially flush with the connector 32. Preferably the head 80 of the screw is round and has a diameter only slightly smaller than the extent of the pressure plate 122. This allows the head 80 of the screw 76 to apply pressure across a wide area of the pressure plate. Means are provided in the screw head for tightening the connector onto the apparatus. For example, two diametrically spaced spanner holes 128, 130 may be provided. Other configurations, such as, for example, slot, star or square patterns, can also be used.

A non-conductive plate 108 fits within the box 86 and supports the connections 94. The plate 108 may be a material such as Mylar. In the preferred embodiment, the connections 94 are female connections, but male pins could also be used in the connector 32, while female connections could be used on the apparatus 12. The wires W1 . . . Wn are connected to the connections by mechanically inserting ends of the wires in slots 110 in sides 112 of the connections 94, by welding, or by other suitable means. A central bore 114 in the plate 108 corresponds to the central bore 90 in the box and provides a passageway for the screw 76. The wires W1 . . . Wn extend through the opening 95 and into the lead 22. With the plate 108 positioned in the box 68, a gasket 116 is placed over the plate 108. The gasket 116 fits over the female connections 94 and the compressible gasket allows the wires W1 . . . Wn to extend from the connections to the lead. A third central bore 120 corresponds to the central bore 114 in the plate 108 and provides a passageway for the screw 76. Behind the gasket 116, a pressure plate 122 fits into the box 86 over the gasket 116. A fourth central bore 124 provides a passageway for the screw 76. A recess 126 surrounds the central bore 124 and allows the head 80 of the screw 76 to be tightened essentially flush with the connector 32. Preferably the head 80 of the screw is round and has a diameter only slightly smaller than the extent of the pressure plate 122. This allows the head 80 of the screw 76 to apply pressure across a wide area of the pressure plate. Means are provided in the screw head for tightening the connector onto the apparatus. For example, two diametrically spaced spanner holes 128, 130 may be provided. Other configurations, such as, for example, slot, star or square patterns, can also be used.

A second embodiment 94b for the female connections 94 is illustrated in FIGS. 12 and 13. This second embodiment is believed to provide a more consistent electrical contact, but may be more difficult to manufacture than the first embodiment. Each female connection 94b of the second embodiment comprises a cylinder 148 having a distal end 150 and a proximal end 152. An outer surface 154 of the cylinder is milled down from the proximal end 152, forming a shoulder 156 between the reduced diameter proximal end 152 and the larger diameter distal end 150. As the female connections are inserted into the plate 108, the shoulder 156 stops the connections at a pre-selected depth. A small diameter bore 158, shown in dotted lines in FIG. 13, is drilled through the cylinder 148. A stopped bore 160 drilled from the proximal end 152 of the cylinder past the shoulder 156 into the distal end 150 forms a first conical side 162 of radially spaced teeth 164. A second, distally-facing, conical side 166 is formed by milling a conical relief into the distal end 150 of the cylinder. Four diametric slots 168, 170, 172, 174 cut across the distal end 150 form eight radially symmetrical tabs 176, 178, 180, 182, 184, 186, 188, 190. Each of the symmetrical tabs carries an inwardly directed tooth 164, formed as described above. The teeth provide a very secure electrical contact for the pins. As in the first embodiment 94a, the slot 110 cut diametrically across the proximal end 152 of the second embodiment 94b receives a wire Wn, which is electrically connected to an electrode or sensor in the lead.

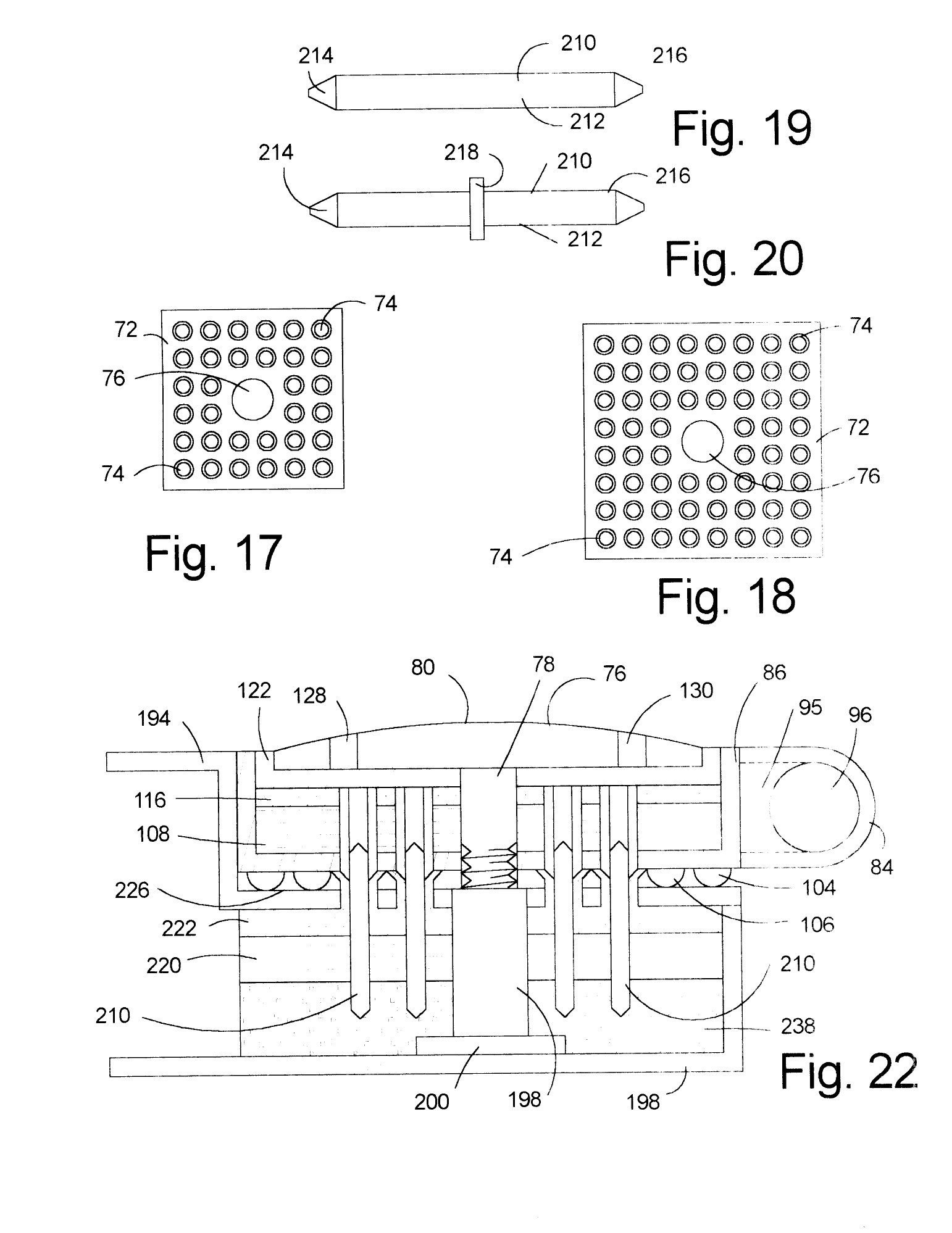

The connector 32 attaches to the apparatus 12 at the recess 64. Preferably, the apparatus 12, such as a cardiac stimulator, comprises electronic circuits and batteries housed in an hermetically sealed case 192. The case 192 comprises a front clam shell 194 and a back clam shell 196 that, when welded together, form the case. As explained above, the bottom wall 72 of the recess 64 has an array of apparatus connections 74 deployed around the threaded bore 75. In the preferred embodiment, the apparatus connections 74 are pins, mounted in a rectangular array as shown, for example in FIGS. 17 and 18. In FIG. 17, an array of thirty-two pins is shown. In FIG. 18, the number of pins has been increased to sixty by adding a set of pins around the perimeter of the array. Similarly, ninety-six or one hundred and twenty connections could be provided. Clearly, the number of electrodes could be adjusted by providing rectangular or asymmetrical arrays. One advantage of the connector of the present invention is that the connections are essentially independent of each other, from a manufacturing stand point. The manufacturing tolerances for any particular connection can be specified from a single point of origin. Adding additional connections does not significantly increase the difficulty of manufacturing, nor do the manufacturing tolerances add together as the number of connections increases.

A column 198 contains the threaded bore 75. The column has a flange 200 on a distal end 202 that can be welded to an interior wall 204 of the back clam shell 196. A proximal end 206 of the column 198 extends through a hole 208 in the front clam shell 194. Tightening the screw 76 in the threaded bore 75 transfers the compressive force holding the connector 32 on the cardiac stimulator 12 to the back clam shell 196. A certain amount of compressive force can be taken by the clam shell in the manner of a spring, thereby providing an additional measure of safety for the seals of the connector 32. The apparatus connections 74 comprise pins, such as the embodiments of FIGS. 19 and 20. A pin 210 may have a metallic, electrically conducting shaft 212, with tapered ends 214, 216, as shown in FIG. 19. If desired, a circumferential flange 218 may be added to provide an assembly stop. The pins 210 are inserted into a non-conducting backing plate 220 until they reach a prescribed insertion, which may be controlled by the presence of the flange 218.

A special gasket 222 fits over the pins 210. As seen in FIG. 21, has a base sheet 224 supporting an array of protrusions 226. Each protrusion 226 has a through bore 228 for a pin 210. An upper edge 230 of the protrusions may be rounded or chamfered 232, particularly adjacent the through bore 228. When the connector 32 is mounted on the apparatus 12, the distal ends of the female connectors 94 will slide along the pins inside the protrusions, as shown in FIG. 22. Thus, there will be three seals separating any electrical connection from body fluids: the two O-rings 104, 106 and a protrusion 226 of the gasket 222. If either the lead or the device is replaced or exchanged, at least one seal will be new at the time of replacement.

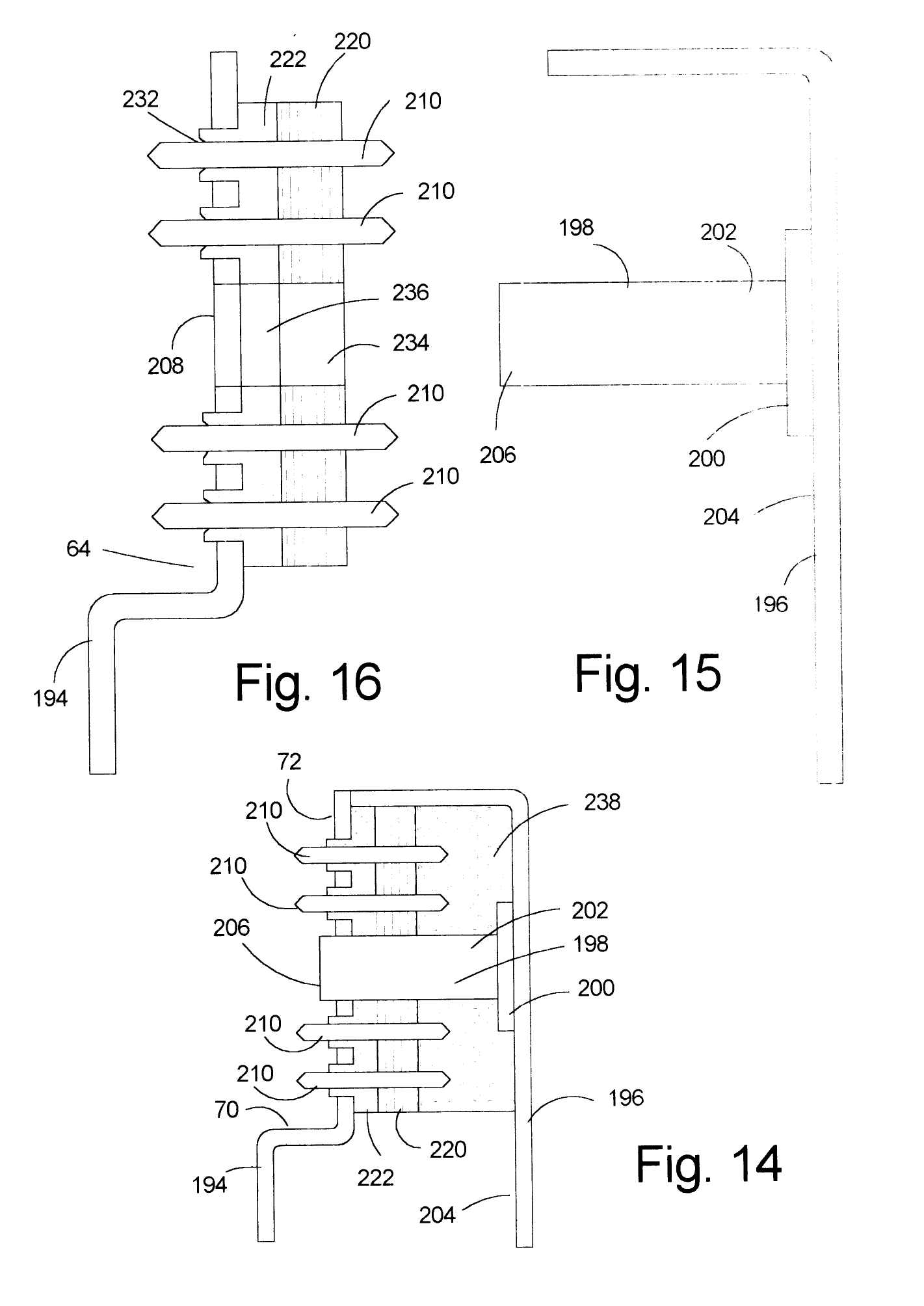

The gasket 222 is sandwiched between the backing plate 220 and the front clam shell 194, as shown in FIG. 16. Central bores 234, 236 in the backing plate 220 and the gasket 222 allow the column 198 to extend from the back clam shell 196 through the hole 208 in the front clam shell 194, as shown in FIG. 14. Electrical connections between the pins 210 and circuits to be mounted inside the case 192 are made by connecting wires (not shown). A filler 238 or other substance may be inserted between the backing plate 220 and the back cam shell 196 to provide additional insulation and sealing around the connections. The filler may also carry compressive force when the connector is mounted on the apparatus 12. The two clam shells are assembled and welded together by laser welding, for example.

The assembly of the connector 32 and apparatus 12 can be seen in cross section in FIG. 22. The spanner screw 76 is inserted through the connector 32 into the threaded bore 75 of the column 198. Pressure of the head of the screw uniformly compresses the two O-rings and the protrusions of the gasket, thereby providing three seals for each connection.

Numerous other modifications may be made to this invention without departing from its scope as defined in the attached claims.

1. An implantable medical apparatus comprising
an electrical device having a case and a plurality of electrical contacts on said case,
a lead having a distal end and a proximal end and carrying a plurality of electrical conductors, and
a connector coupled to a proximal end of said lead, said connector having a central bore a plurality of electrical contacts distributed around said bore, each of said contacts being electrically connected to an electrical conductor in said lead and being spatially arranged to contact one of said electrical contacts on said case, and
a fastener coupling said connector to said electrical device, said fastener extending through said bore in said connector and having a wide head, said head distributing compressive force over said electrical contacts in said connector.

2. The implantable medical apparatus of claim 1 wherein said wide head extends over substantially all said electrical contacts.

3. The implantable medical apparatus of claim 2 wherein said wide head is a disc.

4. The implantable medical apparatus of claim 3 wherein said fastener comprises a threaded shaft.

5. The implantable medical apparatus of claim 2 wherein said contacts around said bore are radially symmetrical around said bore.

6. The implantable medical apparatus of claim 5 wherein said contacts are arranged in a rectangular pattern around said bore.

7. The implantable medical apparatus of claim 6 wherein said contacts are arranged in a plurality of rectangles.

8. The implantable medical apparatus of claim 7 wherein the rectangles are squares.

9. The implantable medical apparatus of claim 1 wherein said case comprises a first side and a second side substantially parallel to said first side and a wall connecting said first and second sides, said first side having a recess therein, said connector fitting into said recess.

10. An implantable medical apparatus comprising
an electrical device having a case and a plurality of electrical contacts on said case,
a lead having a distal end and a proximal end and carrying a plurality of electrical conductors,
a connector coupled to a proximal end of said lead, said connector having a central bore a plurality of electrical contacts distributed around said bore, each of said contacts being electrically connected to an electrical conductor in said lead and being spatially arranged to contact one of said electrical contacts on said case, and
a fastener coupling said connector to said electrical device, said fastener extending through said bore in said connector, and
a plurality of connector seals, each connector seal configured to surround an electrical contact on said case and an electrical contact on said connector when said connectors are in contact with each other.

11. The implantable medical apparatus of claim 10 further comprising a gasket circumscribing said electrical contacts.

12. The implantable medical apparatus of claim 10 wherein at least some of said electrical contacts are male contacts and each of said seals is mounted around a male contact and a corresponding female electrical contact is sliding received between said male contact and said seal mounted around said male contact.

13. The implantable medical apparatus of claim 12 wherein said male contacts are mounted on said case and said female contacts are mounted on said connector.

14. The implantable medical apparatus of claim 13 further comprising a first gasket circumscribing said electrical contacts, said first gasket being mounted on said connector.

15. The implantable medical apparatus of claim 14 further comprising a second gasket circumscribing said first gasket.

16. The implantable medical apparatus of claim 10 wherein said seals are formed on a flexible sheet, said sheet being mounted under a rigid surface and wherein said seals extend through said surface.

17. The implantable medical apparatus of claim 16 wherein said rigid surface is a part of said case and said sheet is inside said case.

18. The implantable medical apparatus of claim 17 further comprising at least one gasket circumscribing said electrical contacts and wherein said at least one gasket is mounted on said connector.

19. The implantable medical apparatus of claim 18 further comprising a fastener centrally located on said connector and wherein said electrical contacts on said connector are symmetrically arranged around said fastener.

20. The implantable medical apparatus of claim 10 further comprising a fastener centrally located on said connector and wherein said electrical contacts on said connector are symmetrically arranged around said fastener.

21. An implantable medical apparatus comprising
an electrical device having a case and a plurality of electrical contacts on said case,
a lead having a distal end and a proximal end and carrying a plurality of electrical conductors,
a connector coupled to a proximal end of said lead, said connector having a plurality of electrical contacts, each of said contacts being connected to at least one of said electrical conductors and being arranged to connect with at least one of said electrical contacts on said case,
at least some of said electrical contacts comprising pins, and
at least some of said electrical contacts comprising tubes having a proximal end having a slot for receiving an electrical conductor and a distal end having a reduced internal diameter for contacting one of said pins.

22. The implantable medical apparatus of claim 21 wherein said distal end of said tubes comprises a plurality of tabs bent inwardly towards a center of said tube.

23. The implantable medical apparatus of claim 21 wherein said distal end comprises at least one diametric slot and a plurality of tabs, each tab having a radially inwardly directed tooth.

24. The implantable medical device of claim 23 wherein said raidally inwardly directed tooth has a proximally-facing conical side and a distally-facing conical side.

25. The implantable medical device of claim 24 wherein said tubes have an outer side with a circumferential shoulder thereon.

26. The implantable medical device of claim 21 wherein said tubes have an outer side with a circumferential shoulder thereon.
